(19) United States
(12) Patent Application Publication
(10) Pub. No.: US 2026/0053627 A1
(43) Pub. Date: Feb. 26, 2026

(54) MEDICAL TRANSDUCER AND METHOD OF DESIGNING WINDING PATTERN OF COIL IN THE MEDICAL TRANSDUCER INCLUDING COIL TOPOLOGY OPTIMIZATION

(71) Applicant: SOLENIC MEDICAL, INC., Addison, TX (US)

(72) Inventors: Rajiv CHOPRA, Dallas, TX (US); Bibin PRASAD, Dallas, TX (US); John TEPPER, Carrollton, TX (US); Luke GRITTER, Delaware, OH (US)

(21) Appl. No.: 19/104,298

(22) PCT Filed: May 22, 2024
(86) PCT No.: PCT/US2024/030467

§ 371 Date: Feb. 17, 2025

Related U.S. Application Data
(60) Provisional application No. 63/503,733, filed on May 23, 2023.

(51) Int. Cl. A61F 2/30; A61F 2/32; A61F 2/38; A61F 2/48

(52) U.S. Cl. CPC A61F 2/30942; A61F 2/482; A61F 2002/30004; A61F 2002/30668; A61F 2002/30952; A61F 2/32; A61F 2/38

(57) ABSTRACT

Embodiments provide a method for determining an alternating magnetic field (AMF) transmitter design that produces a magnetic field that achieves a more uniform surface current or heating of an implant. Embodiments may utilize the method to be applicable to different geometries of implants and AMF transmitters. Embodiments produce a non-uniform magnetic field that in turn produces a more uniform current density/distribution on the implant surface. This leads to uniform heating and consistent biofilm reduction, while minimizing damage to adjacent tissues. Also, the differences in electrical properties such as conductivity, permittivity, and permeability of the implant components can be factored into the design process to achieve a uniform current density/distribution across components. An ultimate benefit derived from embodiments described herein is consistent biofilm reduction, inactivation, eradication, destruction, and/or removal and improved safety arising from heat conduction into surrounding tissues.

### BACKGROUND

A varying magnetic field (VMF) such as an alternating magnetic field (AMF) is a non-invasive approach to treat implant-associated infections, in which an external transducer coil generates time-varying AMF in the vicinity of an electrically conductive implant such as a metal implant in the body. The AMF generates surface electrical currents on the implant, which leads to heating restricted to the surface of the implant. In the case of an infected implant, bacteria, which may be in the form of a biofilm, adhere to the surface within this heated range. This localized heating can be used to eradicate pathogens or sensitize them to antimicrobial treatment.

### BRIEF DESCRIPTION OF THE DRAWINGS

Features and advantages of embodiments of the present invention will become apparent from the appended claims, the following detailed description of one or more example embodiments, and the corresponding figures. Where considered appropriate, reference labels have been repeated among the figures to indicate corresponding or analogous elements.

FIG. 1 includes an example prosthetic knee implant geometry.

FIG. 2 includes a half-symmetric model with source current on cylindrical shell.

FIG. 3 includes a noisy distribution of ψ.

FIG. 4 depicts magnitude of surface current density Jₛ and FIG. 5 depicts its corresponding vector field.

FIG. 6 depicts magnitude of the induced current density (also known as current distribution) on the surface of the implant.

FIG. 7 depicts equipotential contours with (a) 100 A spacing and (b) 200 A spacing between loops.

FIG. 8 depicts edge coils generated with 100 A in each loop (half-symmetric).

FIG. 9 provides a comparison of induced current density distribution produced by (a) discrete edge loops and (b) optimized current sheet.

FIG. 10 depicts a flow chart depicting a process in an embodiment.

FIGS. 11(A)-11(F) depict coil designs using the forward method. As a result, heating is non-uniform on implant. FIG. 11(A) is a Curved Pancake Coil. FIG. 11(B) is an Array Coil. FIG. 11(C) is a Helmholtz Coil. FIG. 11(D) is a Solenoid coil. FIG. 11(E) depicts non-uniform heating on implant from a Helmholtz coil. FIG. 11(F) depicts non-uniform heating on implant from a Solenoid coil.

FIG. 12 illustrates a horse-shoe coil shape to fit easily around a knee.

FIGS. 13(A) and 13(B) illustrate the Optimization Method whereby one builds a numerical model of the surface for currents (horseshoe) and the implant to be exposed to AMF. FIG. 13(A) shows a horseshoe surface for coil winding generation.

FIG. 13(B) shows an implant placed inside the surface.

FIGS. 14(A), 14(B), and 14(C) illustrate results from an embodiment of the optimization method that generates a custom coil-winding pattern that achieves a desired current density/distribution and/or heating on the implant surface. FIG. 14(A) is a coil winding pattern. FIG. 14(B) shows surface current density/distribution from the coil and FIG. 14(C) shows heating from the coil.

FIG. 15(A) shows a 3D CAD model created from optimization winding. FIG. 15(B) shows a 3D printed coil from the CAD model.

FIGS. 16(A)-16(E) address comparisons between magnetic fields from different coil designs. FIG. 16(A) shows a field from a Solenoid coil. FIG. 16(B) shows a field from a Hemholtz coil. FIG. 16(C) shows a scale for FIGS. 16(B) and 16(D)-16(E). FIGS. 16(D) and 16(E) show a field for an optimized coil.

FIGS. 17(A)-17(G) address comparisons between surface current density/distribution and heating from different coils thereby illustrating the improved uniformity achieved with the optimization approach. FIG. 17(A) shows a scale for FIGS. 17(B)-17(G). FIGS. 17(B) and 17(E) respectively show current density/distribution and heating for a Solenoid coil. FIGS. 17(C) and 17(F) respectively show current density/distribution and heating for a Helmholtz coil. FIGS. 17(D) and 17(G) respectively show current density/distribution and heating for an optimized coil.

FIG. 18 is a flow chart schematically depicting a process of designing the coil winding pattern to generate varying magnetic fields (VMF) from a medical transducer in exemplary embodiments.

FIG. 19 illustrates results from an embodiment of the process of designing the coil winding pattern such as the optimization method that generates a custom coil-winding pattern of a first coil within a first side compartment of a medical transducer for a hip implant that achieves a desired current density/distribution and/or heating on the implant surface.

FIG. 20 illustrates results from the embodiment of FIG. 19 that generates a custom coil-winding pattern of a second coil within a second side compartment of the medical transducer for the hip implant that achieves a desired current density/distribution and/or heating on the implant surface.

FIG. 21 includes a system for use with an embodiment of the invention. FIG. 22 includes a system for use with an embodiment of the invention. FIG. 23 includes a system for use with an embodiment of the invention.

FIG. 24 includes a flow chart depicting a process in an embodiment.

### DETAILED DESCRIPTION

Reference will now be made to the drawings wherein like structures may be provided with like suffix reference designations. In order to show the structures of various embodiments more clearly, the drawings included herein are diagrammatic representations of structures. Thus, the actual appearance of the fabricated structures, for example in a photo, may appear different while still incorporating the claimed structures of the illustrated embodiments (e.g., walls may not be exactly orthogonal to one another in actual fabricated devices). Moreover, the drawings may only show the structures useful to understand the illustrated embodiments. Additional structures known in the art may not have been included to maintain the clarity of the drawings. For example, not every layer of a device is necessarily shown. “An embodiment”, “various embodiments” and the like indicate embodiment(s) so described may include particular features, structures, or characteristics, but not every embodiment necessarily includes the particular features, structures, or characteristics. Some embodiments may have some, all, or none of the features described for other embodiments. “First”, “second”, “third” and the like describe a common object and indicate different instances of like objects are being referred to. Such adjectives do not imply objects so described must be in a given sequence, either temporally, spatially, in ranking, or in any other manner. “Connected” may indicate elements are in direct physical or electrical contact with each other and “coupled” may indicate elements co-operate or interact with each other, but they may or may not be in direct physical or electrical contact. Phrases such as “comprising at least one of A or B” include situations with A, B, or A and B.

An embodiment provides a medical transducer and a method of designing winding pattern of coil in the medical transducer including coil topology optimization. In certain embodiments, Applicant determined the desired exposure using AMF is to generate uniform surface electrical currents or heating on the infected implant. Most medical implants (e.g., a knee implant) are asymmetric in all dimensions (possibly employing one or more different materials), and simple magnetic field transmitters (e.g., solenoid, Helmholtz, loop coil) do not produce uniform heating of the implant. Applicant determined this can lead to overheating or underheating of the implant and either inadequate infection removal or excess tissue damage. In other words, exposing an implant to a conventional uniform magnetic field results in a very non-uniform current density pattern on the implant, which in turn leads to non-uniform heating and ultimately heterogeneous treatment of biofilm. Complicating matters further is the fact that the different components of an implant often have different electrical properties since they are made from different alloys, which results in different current densities/distribution and rates of heating for the individual components.

### 1. Objective

Embodiments, however, provide a method for determining an AMF transmitter design that produces a magnetic field that achieves a more uniform surface current or heating of an implant. Embodiments may utilize the method to be applicable to different geometries of implants and AMF transmitters. In other words, the described embodiments overcome the above challenges by producing a non-uniform magnetic field that in turn produces a more uniform current density/distribution on the implant surface. This leads to uniform heating and consistent biofilm reduction, while minimizing damage to adjacent tissues. Also, the differences in electrical properties such as conductivity, permittivity, and permeability of the implant components can be factored into the design process to achieve a uniform current density/distribution across components. While permittivity measures the ability of the implant to store energy within the implant, permeability is a measure of the ability of the implant to support the formation of a magnetic field within the implant. An ultimate benefit derived from embodiments described herein is consistent biofilm reduction, inactivation, eradication, destruction, and/or removal and improved safety arising from heat conduction into surrounding tissues. A detailed review of embodiments now follows.

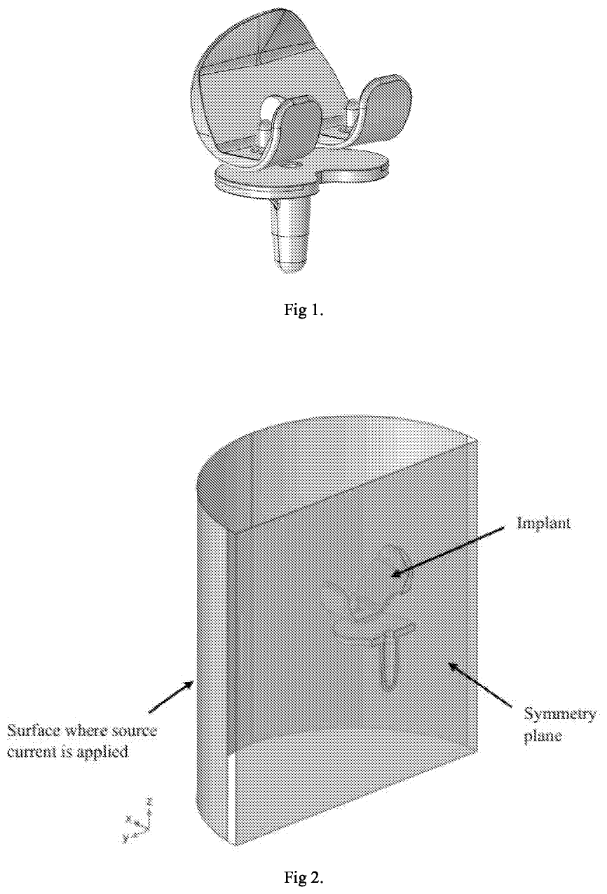

A tailored alternating magnetic field is applied to an electrically conductive implant (FIG. 1) to induce a specified current density/distribution or heat source distribution within the implant. This magnetic field is produced by a varying electric current (e.g., an alternating electric current) flowing through a set of coil windings, and the design of the coil windings is optimized to produce the desired effect on the implant.

### 1.1 Objective Function

Generally, the target current density/distribution or heat source distribution will not be perfectly achievable at every location in the implant, so the goal of the coil design process is to find the winding pattern that comes nearest to producing the desired effects in the implant while also satisfying any necessary constraints. To enable comparison of the performance of different coil designs, an objective function is defined to quantify how well a design meets the specified target, and the optimal surface current function either minimizes or maximizes this objective function, depending on the manner in which the objective function is defined.

For example, if the desired effect is to produce a specified current density distribution J_{target}(x) on the surface of the implant, the objective function could be defined as a normalized root-mean-square:

$${f}_{R M S} ≡ {[ \frac{\int {\int }_{S} {( J ( x ) - {J}_{target} ( x ) )}^{2} dS}{\int {\int }_{S} d S} ]}^{0.5} [ \frac{\int {\int }_{S} d S}{\int {\int }_{S} {J}_{t a r g e t} ( x ) d S} ] \qquad ( 1 )$$

where J(x) is the magnitude of the current density/distribution at location x and S represents the surface of the implant. A lower value of the objective function f_{RMS} represents a design that more closely matches the target current density distribution. To include the effects of constraints in the objective function, penalty terms can be added to rapidly increase the objective function when a constraint is breached. As will be explained in more detail, the constraints may include, but are not limited to, maximum and minimum allowable current densities, current amplitude range for a coil, current direction within a coil, spacing between adjacent portions of a coil, proximity to critical tissues such as nerves, among others. If the implant components are made of different materials, different targets on the components can be achieved using the objective function.

### 1.2 Approach for Designing Coils

The optimal coil winding pattern will depend on the shape and material properties of the implant as well as the desired current density/distribution or heat source distribution, and the topology of an optimal winding pattern is typically not intuitive. To design an appropriate winding pattern, a physics-based simulation is developed and combined with the following three techniques:
1) Instead of exciting the magnetic field by a current flowing through a discrete winding pattern, the simulation uses a continuous source current density distribution within a specified volume or on a specified surface.
2) An optimization algorithm is utilized to find the source current density distribution that minimizes the objective function.
3) The optimized source current density distribution is discretized into a winding pattern that produces approximately the same magnetic field distribution.

The combination of a continuous source current distribution with an optimization algorithm enables an automated exploration of an unlimited variety of coil topologies, and the discretization step produces a buildable coil design from the optimized current source. The physics-based simulation may be conducted using, for example, finite-element simulation software COMSOL Multiphysics (Comsol v5.6, Los Angeles, CA, USA).

### 2. Source Current

The interaction of the magnetic field with the implant is modeled using the Maxwell-Ampere equation in the frequency domain:

$$∇ \times H = σ E + j ω D + {J}_{e} \qquad ( 2 )$$

where H is the magnetic field, σ is the electrical conductivity, E is the electric field, ω is the angular frequency, D is the displacement field, and Jₑ is an external source current. In this application, Jₑ is the source current density distribution on the transducer that is being optimized to produce the desired effects in the implant.

### 2.1 Divergence-Free Current Source

For the source current density distribution to represent a physically realizable state, it must satisfy the stationary current conservation equation, which means that it must be divergence-free:

$$∇ \cdot {J}_{e} = 0 \qquad ( 3 )$$

If the Jₑ distribution is directly specified by the optimization solver, equation 3 will generally not be satisfied. To ensure that Jₑ is always divergence-free, it is not specified directly but calculated from a vector potential 4P according to the following equation:

$$∇ \times Ψ = {J}_{e} \qquad ( 4 )$$

Since the divergence of the curl of a vector is always zero, the divergence of Jₑ is zero for any vector ψ:

$$∇ \cdot {J}_{e} = ∇ \cdot ( ∇ \times Ψ ) = 0 \qquad ( 5 )$$

The optimization process is performed on ψ instead of Jₑ, ensuring that only physically allowable distributions of Jₑ are considered.

### 2.2. Shell Approximation of Current Source

To reduce the size of the optimization problem and facilitate the generation of a buildable coil topology, the source current distribution can be applied on a collection of surfaces (a shell) rather than in a volume. In this case, the Jₑ term in the Maxwell-Ampere equation is replaced by a boundary condition on the shell:

$$n \times ( {H}_{1} - {H}_{2} ) = ( n \times {J}_{s} ) \times n \qquad ( 6 )$$

The magnetic field is discontinuous across the shell, and H₁ and H₂ in equation 6 are the magnetic field on each side of the shell. The vector Jₛ is the applied surface current density on the shell, and n is the surface normal vector. The shape of the shell can either be treated as a fixed model input or be solved for as a part of the optimization process. FIG. 2 illustrates a half-symmetric model where the source current is applied on a cylindrical shell. The shell can be of any shape and size that is consistent with the end use of the device.

As with the volume current source, the surface current source also must be divergence-free to be physically realizable, and a similar method is used to ensure that this requirement is fulfilled. In this case, however, the vector potential can be replaced by the product of a scalar potential p and the surface normal vector:

$$∇ \times ( ψ n ) = {J}_{s} \qquad ( 7 )$$

This formulation has three benefits:
1. The use of a scalar potential rather than a vector potential reduces the number of optimization control variables by a factor of three, speeding up the optimization process.
2. The current density vector is orthogonal to the surface normal vector and therefore tangential to the shell surfaces, which is consistent with a winding pattern that lies on the shell.
3. Equipotential lines represent current loops, facilitating the generation of winding patterns (see section 4.1)

To maintain the global conservation of current on the shell, Jₛ is constrained to be tangential to the edge at the outer edges of the shell:

$$t \times {J}_{s} = 0 \qquad ( 8 )$$

where t is the edge tangent unit vector. For a continuous surface, ∇×n=0, so equation 7 can be rewritten as:

$${J}_{s} = ∇ \times ( ψ n ) = ψ ∇ \times n + ( ∇ ψ ) \times n = ( ∇ ψ ) \times n \qquad ( 9 )$$

and equation 8 can then be rewritten as

$$t \times [ ( ∇ ψ ) \times n ] = 0 \qquad ( 10 )$$

This condition is fulfilled if ψ is set to a constant value around the outer edges of the shell.

### 3. Optimization Method

The mathematical model of the physical system can be solved using any suitable method, such as the finite element method, and this solution process is embedded in an optimization routine that seeks to find the optimal source current distribution on the shell by minimizing an objective function such as f_{RMS} in equation 1. To enable the optimization solver to adjust the source current distribution, control variables can be applied at each node point in the finite element mesh along the shell. These control variables represent the value of ψ at each node point, and the value of ψ between the node points can be interpolated using standard finite element techniques.

### 3.1 Optimization Solver

Since control variables are applied at every node point in the finite element mesh on the shell, the number of control variables can become quite large. To efficiently optimize a large number of control variables, a gradient-based optimization solver must be used, and the Sparse Nonlinear Optimizer (SNOPT) gradient-based optimization solver has been found to work well for this application. The solver typically requires 30 to 50 iterations to minimize the objective function.

The optimization space can be reduced by imposing upper and lower limits on the value of ψ. As these limits are narrowed, the optimization solver typically requires less iterations and produces simpler winding patterns, but the final value of the objective function increases (i.e., the optimized solution moves further away from the target), so the limits are preferably set to gain the advantage of a simpler winding pattern without significantly compromising the ability of the coil to produce the desired effect in the implant. When ψ is constrained to a value of zero at the outer edges of the shell, suitable limits for this application commonly fall in the range of +/−500 A to +/−3000 A, depending on the model inputs.

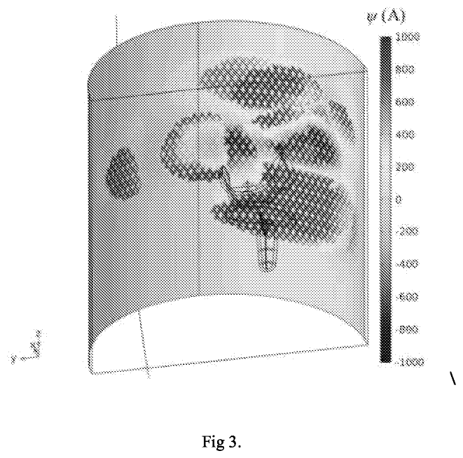

For a cylindrical shell around the implant geometry shown in FIG. 1, the optimization process produces the ψ distribution shown in FIG. 3 when the objective function in equation 1 is used with a constant target current density of 100 A/cm².

### 3.2 Optimized Current Density Distribution

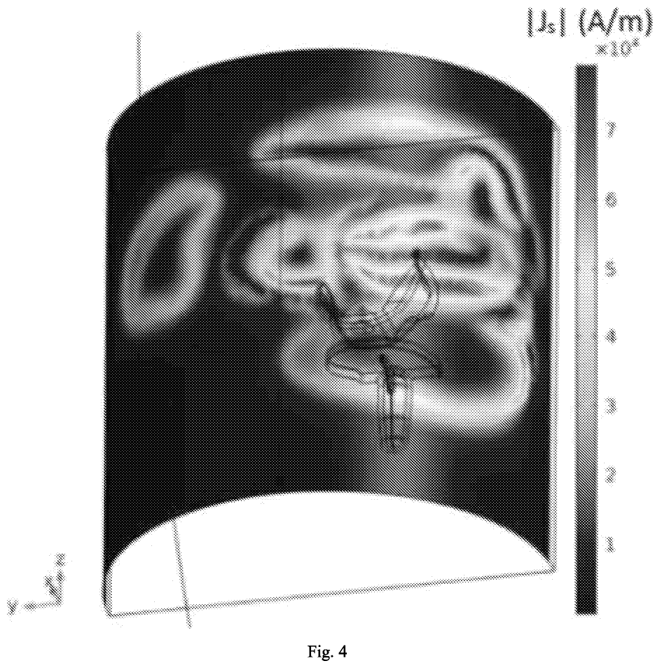

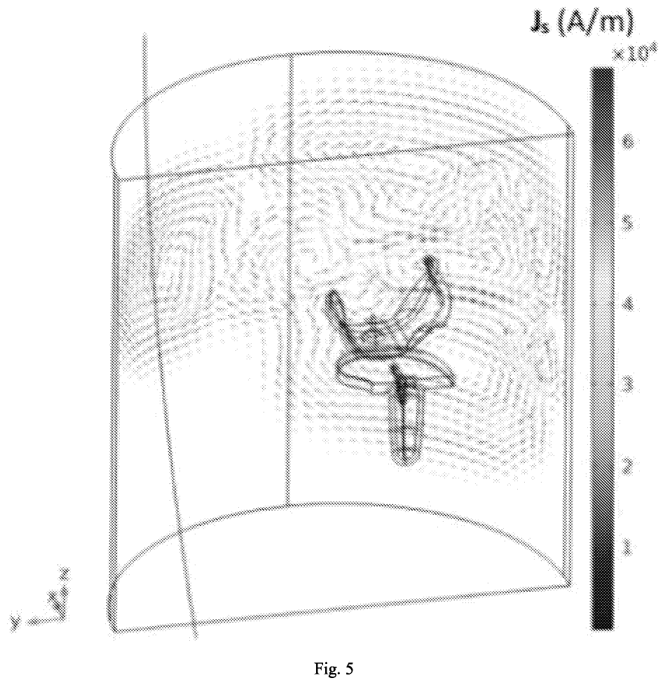

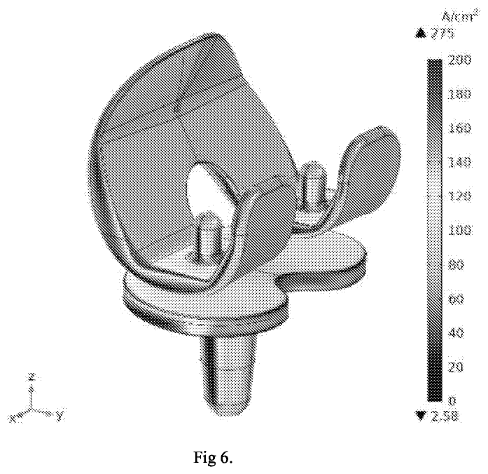

An optimized source current density distribution is shown in FIGS. 4 and 5. This current sheet generates a magnetic field that in turn induces a current in the implant. FIG. 6 shows the induced current density/distribution on the surface of the implant, which is within +/−30% of the target value of 100 A/cm² on about 80% of the implant surface. Further improvement can be achieved by optimizing the shape of the shell on which the source current is applied.

### 4. Discretization of the Current Sheet into Winding Patterns

The optimization procedure produces a source current density distribution on the shell, but it is generally not possible to directly impose such a current distribution in a real system. Instead, the current distribution can be approximated by one or more coils with suitable winding patterns.

### 4.1 Equipotential Lines as Current Loops

In the same manner as shown in equation 9, the source current density distribution is then calculated from equation 11:

$${J}_{s} = ( ∇ {ψ}_{f} ) \times n \qquad ( 11 )$$

for any continuous surface. This expression indicates that Jₛ is not only orthogonal to the surface normal vector (i.e., it is tangential to the surface), but it is also orthogonal to the gradient of ψ_{f}, so that

$${J}_{s} \cdot ∇ {ψ}_{f} = 0 \qquad ( 12 )$$

Consequently, the Jₛ vector is aligned with the direction of constant ψ_{f} at every point, and the current follows the contour lines of ψ_{f}. Since ψ_{f} is constrained to a constant value around the outer edges of the shell, these equipotential contour lines form loops on the shell surfaces.

### 4.2 Loop Number and Spacing

The source current sheet can be approximated by a set of current loops on the shell, and the spacing between the loops is directly related to the amount of current required in each loop. The scalar potential ψ_{f} has units of amperes, and an area on the shell bounded by two contour lines of ψ_{f} carries a current equal to the difference between the values of ψ_{f} on the bounding contour lines. For example, an area bounded by the contour lines ψ_{f}=200 A and ψ_{f}=300 A carries 100 A of current, and the current in this area could be approximated by a single current loop carrying 100 A located on the contour line ψ_{f}=250 A.

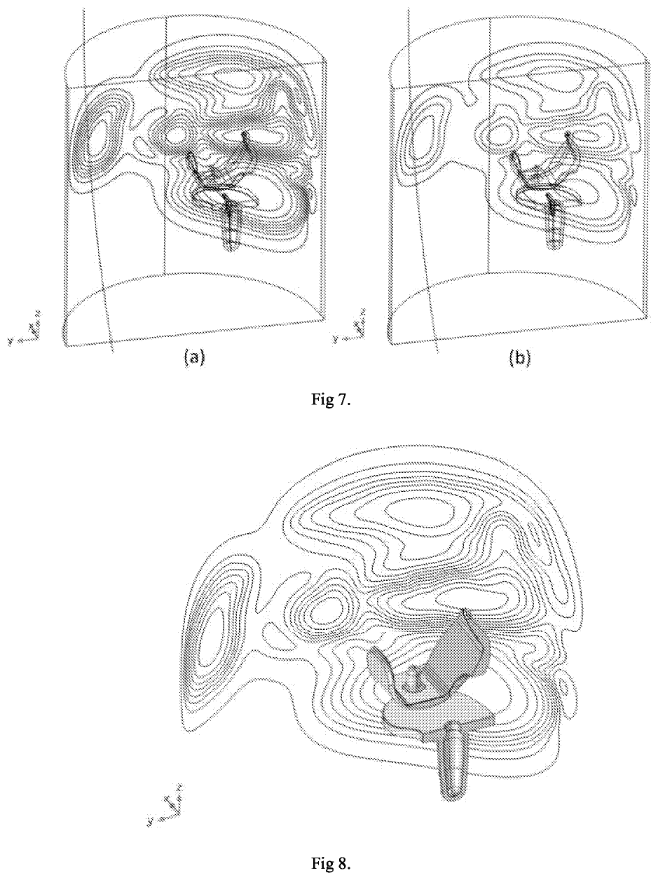

Generally, a larger number of current loops produces a magnetic field distribution that is closer to what is generated by the continuous current sheet. In most cases, however, the effects of the current sheet can be well approximated by a winding pattern made up of less than ten ψ_{f} contour lines. FIG. 7 illustrates two different winding patterns generated from the same optimized source current sheet: (a) 14 ψ_{f} levels with 100 A through each loop and (b) ψ_{f} levels with 200 A through each loop.

When choosing the number of loops to include in the winding pattern, there are several considerations:
1) The magnetic field distribution generated by the discretized winding should be similar to the field generated by the optimized source current sheet to the extent that approximately the same effects are produced in the implant.
2) In a real device, the loops can be connected in series, in parallel, or any combination thereof. In an embodiment, the loops are connected in series. As the complexity of the topology of the winding pattern increases, it can become increasingly difficult to construct a physical coil.
3) The wires or traces making up the coil have finite widths and require gaps of finite thickness between them to maintain electrical isolation. Sufficient spacing between the loops must be maintained to accommodate these spatial requirements.
4) Depending on the cross-sectional area of the wires or traces, the current will need to be limited to a certain value to avoid unacceptable electrical losses or heating in the coil.

To simultaneously fulfill these last two requirements while also achieving the necessary amount of current dictated by the loop spacing, the thickness of the conducting material in the direction normal to the shell surfaces can be increased. For windings constructed with wires, this can be achieved by stacking loops. If the windings become too thick, however, the behavior of the coils will begin to diverge from the optimized source current sheet.

### 4.3 Evaluating the Performance of the Discretized Winding Pattern

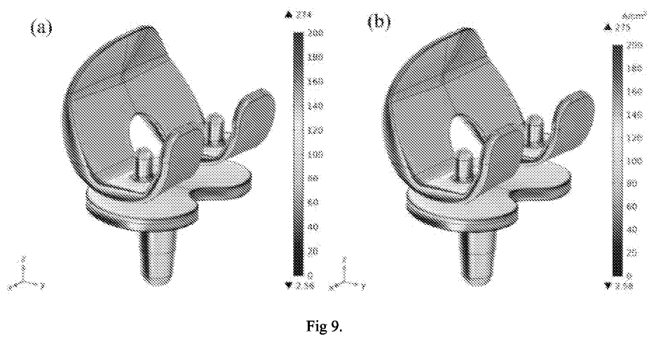

To verify that a chosen set of current loops reproduces the performance of the optimized source current sheet, the shell can be replaced by a set of edge loops generated from the appropriate (pf contour lines, with these edge loops approximating the traces or wires that would be used in a physical system. An example of this is shown in FIG. 8. When 100 A at 200 kHz is applied to each of the edge loops in this example, the resulting induced current density/distribution along the surfaces of the implant is shown in FIG. 9a. FIG. 9b shows the induced current density/distribution for the optimized source current sheet, which is nearly identical to the results generated by the edge loops.

### 5. Flowchart of Optimization Process

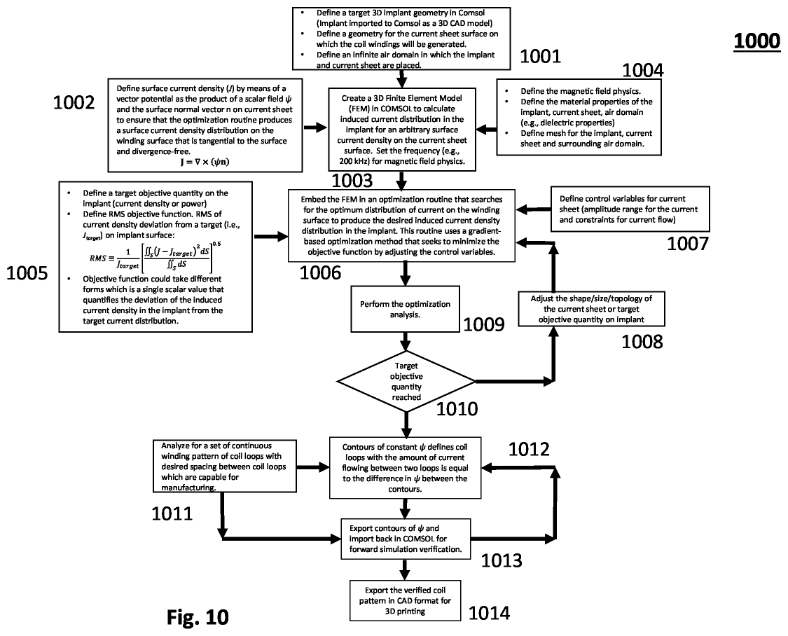

An optimization process (1000) is summarized in the flowchart of FIG. 10.

In block 1001 a user defines the implant's geometry/shape in 3D finite element analysis (modeling and simulation) software (e.g., COMSOL, ANSYS, or similar). This may occur via importation of a 3D CAD file that defines the implant properties (shape and materials) or by the user defining the implant shape and materials within the software. In addition, the user defines the “sheet surface” geometry/shape on which windings will eventually be placed. For example, see the implant and surface of FIG. 2. The “source current” of FIG. 2 will induce the magnetic field on the implant. The source current will eventually be physically realized with windings on the surface to induce a magnetic field on the implant. Finally, the user will define the domain in which the implant and coil surface are placed. This may include the space between the coil surface and implant. By defining the air domain the user will complete the model for simulations. The electromagnetic properties of tissue (bone, skin, and muscle) vary with frequency. The current density generated on the implant surface due to a magnetic field does not vary much if it is in air or tissues. Defining the air domain is addressed further in the discussion for block 1004.

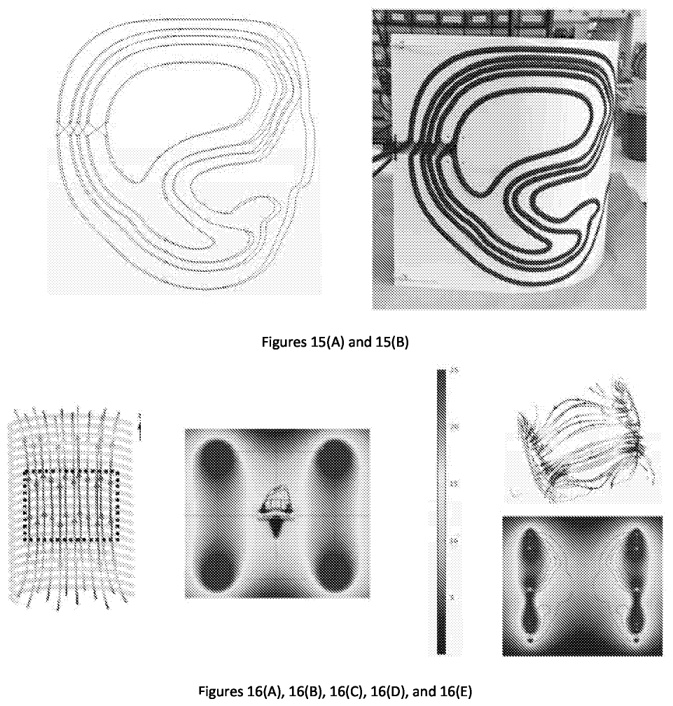

In block 1003 the user defines a finite element model (FEM). Because block 1003 occurs after block 1001 in the flow chart, the acts of block 1003 are “in response to” or “based on” the acts of block 1001. The FEM may include a mesh for the implant, coil surface, and surrounding air domain, where the mesh will be used to determine induced current distribution on the implant. The mesh is addressed further in block 1004. Block 1003 addresses an “arbitrary surface current density”. In this context, “arbitrary” means a shape or pattern that is, for example, not necessarily symmetric about an axis. The knee implant of FIG. 1 may be said to be “arbitrary” due to its irregular profile. Further, the coil pattern of FIG. 15(B) may be said to be “arbitrary” due to its irregular profile wherein varying portions of the coil have different winding densities than other portions of the coil. The FEM of block 1003 is based on inputs from blocks 1002 and 1004.

In block 1002 the user establishes a continuous source current density distribution within a specified volume or on a specified surface, as the product of a scalar field and the surface normal vector, which is a vector potential. This vector potential ensures that the optimization routine produces a current density that is tangential to the surface defined in Block 1001, and divergence free. Defining the current density in this manner is what enables the current density to be realized as coil windings on the surface.

In other words, defining the current density tangential to the surface and divergence free means that the current does not enter or exit either the face or edges of the defined surface, and is therefore physically realizable.

In block 1004 the user makes various definitions including dielectric values for the coil surface, implant, and air domain in which the coil surface and implant are located.

In block 1006 the user may embed the FEM in an optimization routine. This routine will help determine the best coil pattern to generate the desired current density/distribution on the implant. The implant current density is defined in block 1005. This optimization routine uses a gradient-based optimization method that seeks to minimize the objective function by adjusting the control variables.

In block 1005 it is important to note than in conventional forward modeling the end result (e.g., desired current density/distribution on the implant) is not known. However, in this embodiment the end result is defined and then this method works “backwards” (inversely) from that end result. This enables the analytical definition of the magnetic field needed to induce the defined current on the surface. Thus, block 1005 is used to define the ideal end result and acceptable variance/deviation from that result. The RMS optimization is a preferred optimization method, but others are possible. The objective function may be (or may generate) a single scalar value that quantifies the deviation of the induced current density/distribution in the implant from the target current distribution.

In block 1007 the user defines variables for the coil sheet such as current amplitude range and constraints such as current direction and current value of the spacing between the contour lines.

In block 1009 the user defines the frequency for the magnetic field physics based on the material properties of the specific implant. It is important to select a frequency that optimizes the skin effect for the material of the implant. Further, the code performs the optimization analysis based on the RMS objective function defined in block 1005.

In block 1008 the user may define the number of iterations for the optimization analysis of block 1009 to, for example, better manage computing resources.

In block 1010 an iterative process loops back to block 1006 to continue analysis of potential coil patterns from the optimization steps of blocks 1006, 1009 (while observing the iteration limitations of block 1008) until the target objective quantity defined in Block 1005 is reached. If the target objective quantity is not reached, the process adjusts shape, size, and/or topology of the current sheet surface.

In block 1011 the user selects a continuous winding pattern based on the constant current contours from block 1012.

In block 1012 the output of computer analysis is a set of constant current contours which are analyzed in block 1011 to become the winding patterns of coil loops.

In block 1013 The contours selected are re-imported into the finite element modeling software for forward simulation to verify that the implant heating from the selected coil loops is as uniform as desired.

In block 1014 an output may be produced. For example, an optimized coil format may be output as a CAD file, which can then be used to generate the physical coil shown at, for example, FIG. 15(B).

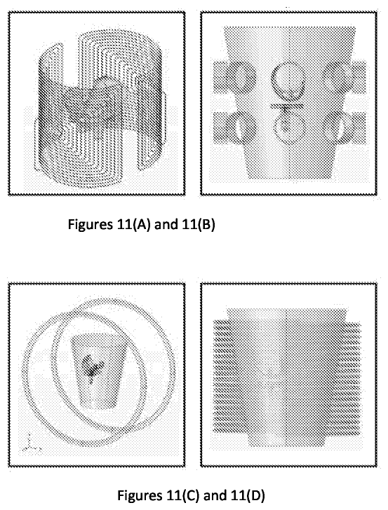

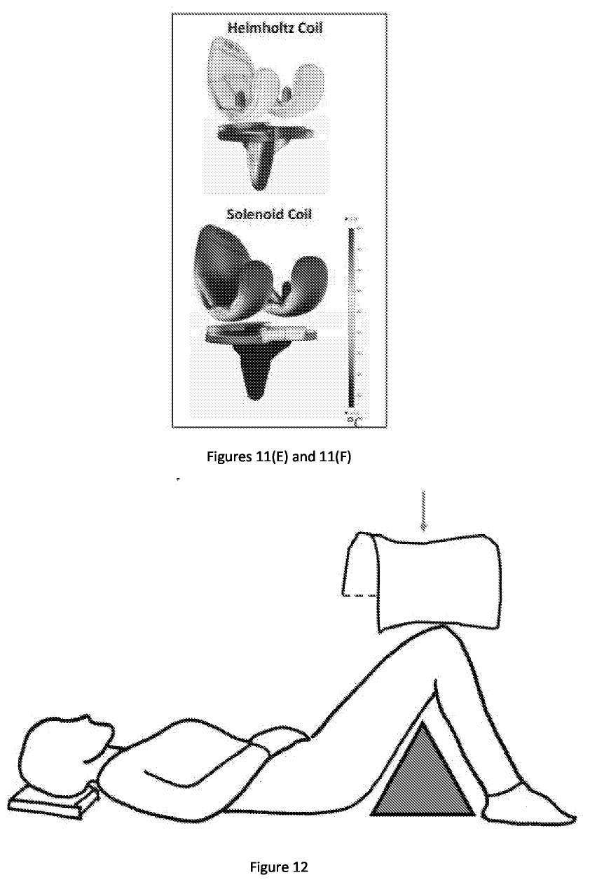

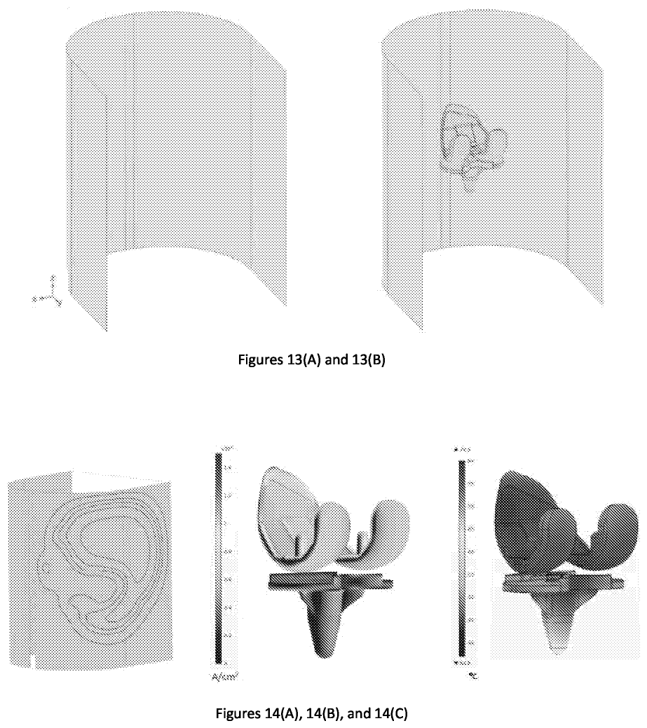

Thus, embodiments provided herein include a method to design and fabricate a coil that induces surface currents (and thereby heat) that are optimally/maximally uniform on the surface of an arbitrarily shaped implant. For instance, FIGS. 11(A)-1i1(D) show conventional coils and how they produce inconsistent heating (FIGS. 11(E)-11(F)) on arbitrarily shaped implants, such as knee implants. These conventional coils were generated as part of a “forward method”, which is basically a “best guess” based on developer intuition, followed by evaluation and analysis to determine if the uniformity of heating is satisfactory. Based on the significant number of coil configurations tried, Applicant determined there was no reasonable way to optimize a coil configuration using the forward method, and as well as no reasonable way to know if an optimum condition was being approached.

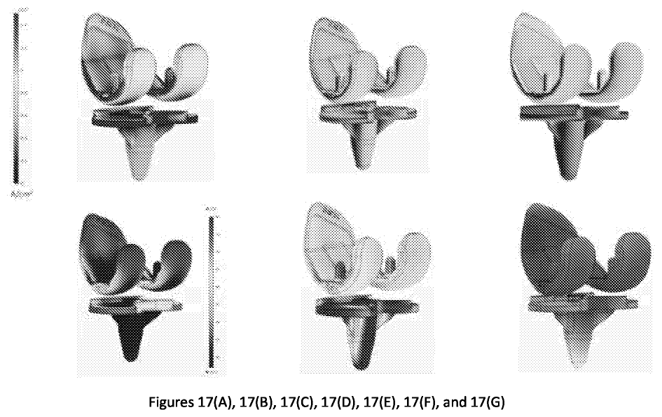

In contrast, embodiments described herein model coils on arbitrary irregularly shaped surfaces (e.g., horseshoe surface of FIG. 12) to generate consistent and even heating on arbitrary irregularly shaped implants. Such embodiments do so using an “optimization method” (such as an RMS objective function). Such a methodology produces better current distribution and, consequently, more even heating along the implant as shown in, for example, FIGS. 14(A)-14(C), 16(D)-16(E), 17(D), 17 (G).

For example, “An Inverted Process for the Streamlined Design of Electromagnetic Coils”, Kline et al., Proceedings of the 2019 COMSOL Conference, Boston, MA. 2019, provides a method for generating a “specified magnetic field”. The method generates a constant field in space. In contrast, embodiments described herein generate a field that specifically varies through space to better induce consistent heating on an irregularly shaped implant. In other words, embodiments specifically establish a specified surface current density in order to generate uniform heating on a conductive object within the magnetic field (which means the magnetic field generated is, in the general case, (very) non-uniform). Embodiments provide uniform heating of the implant using current that is not necessarily uniform, but which generally promotes uniform heating over time. The constant field of Kline would not generate uniform currents and resultant heating on an irregularly shaped implant.

Also, with the Kline methodology, to form full current loops there is a mirroring process which depends on the symmetry of the model. However, embodiments described herein do not depend on symmetry for the target implant and, in fact, readily accommodate irregularly shaped implants because the method includes defining the surface current density required for the desired heating. The process described in the Kline methodology starts with a relatively uniform magnetic field to work from, rather than the relatively uniform current density (or relatively uniform current distribution) on a non-uniform implant surface.

Thus, Kline describes simple uniform fields, or coils generating one or more uniform fields. However, these magnetic fields are not complex enough to generate uniform exposure of a knee or other orthopedic implant. In contrast, embodiments addressed herein create complex 3D magnetic fields that achieve a desired current density/distribution uniformity on a metal implant.

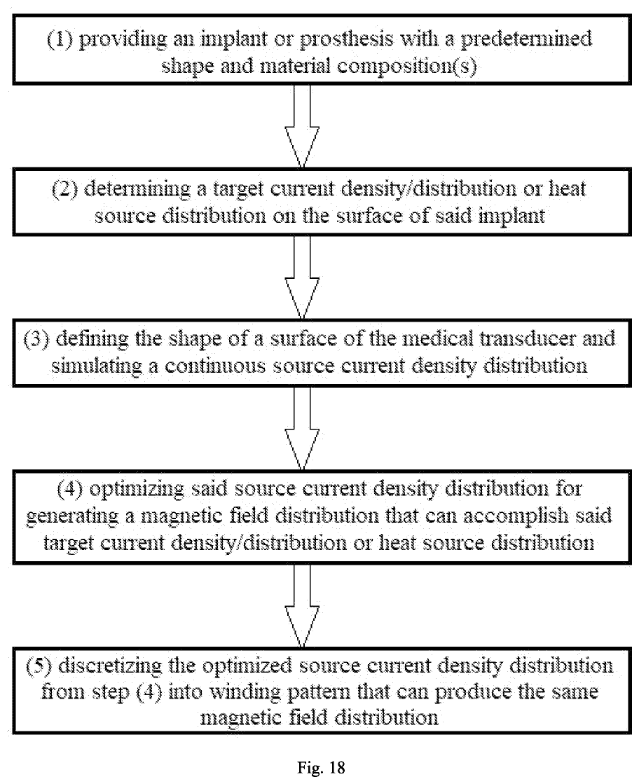

FIG. 18 is a flow chart schematically depicting a process of designing the coil winding pattern to generate VMFs from a medical transducer in exemplary embodiments. In various embodiments, the method of designing the coil winding pattern to generate VMFs such as AMFs from a medical transducer includes: (1) providing an electrically conductive implant or prosthesis (hereinafter “implant”) with a predetermined shape and predetermined material composition(s) throughout said implant; (2) determining a target current density/distribution or heat source distribution across the surface of said implant; (3) defining the shape of a surface of the medical transducer and simulating a continuous source current density distribution; (4) optimizing said source current density distribution of the medical transducer for generating a magnetic field distribution that can accomplish said target current density/distribution or heat source distribution on said implant; and (5) discretizing the optimized source current density distribution from step (4) into a winding pattern that can produce approximately the same magnetic field distribution.

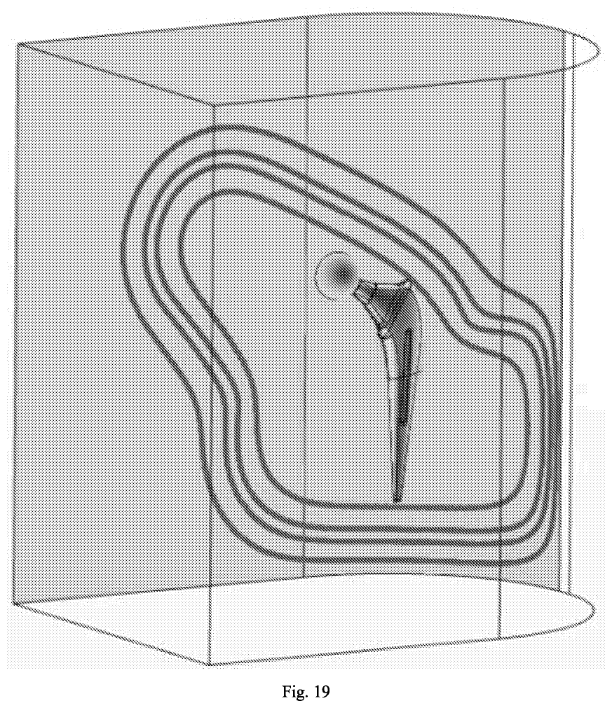

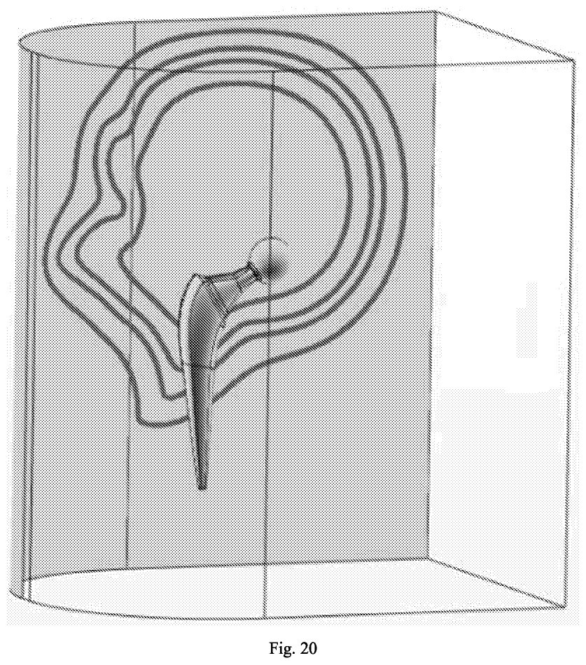

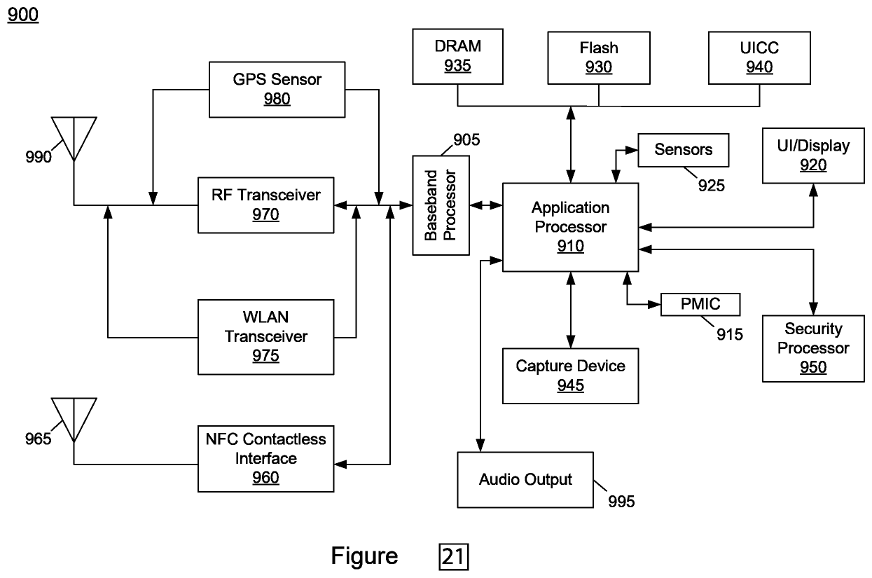

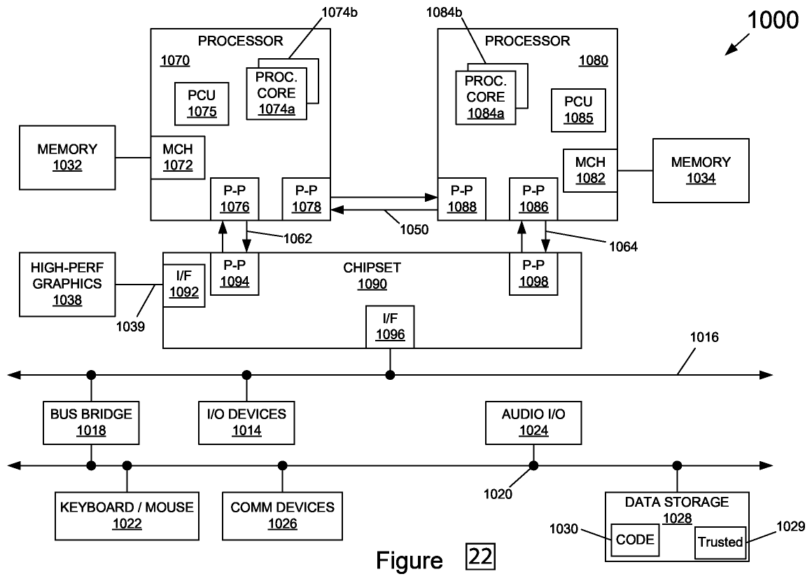

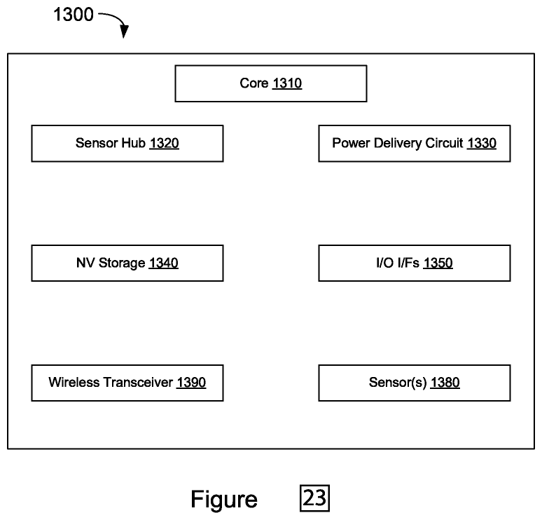

FIG. 19 illustrates results from an embodiment of the process of designing the coil winding pattern such as the optimization method that generates a custom coil-winding pattern of a first coil within a first side compartment of a medical transducer for a hip implant that achieves a desired current density/distribution and/or heating on the implant surface.

FIG. 20 illustrates results from the embodiment of FIG. 19 that generates a custom coil-winding pattern of a second coil within a second side compartment of the medical transducer for the hip implant that achieves a desired current density/distribution and/or heating on the implant surface.

FIG. 21 includes a block diagram of an example system with which embodiments can be used. As seen, system 900 may be a smartphone or other wireless communicator or any other Internet of Things (IoT) device. A baseband processor 905 is configured to perform various signal processing with regard to communication signals to be transmitted from or received by the system. In turn, baseband processor 905 is coupled to an application processor 910, which may be a main CPU of the system to execute an OS and other system software, in addition to user applications such as many well-known social media and multimedia apps. Application processor 910 may further be configured to perform a variety of other computing operations for the device.

In turn, application processor 910 can couple to a user interface/display 920 (e.g., touch screen display). In addition, application processor 910 may couple to a memory system including a non-volatile memory, namely a flash memory 930 and a system memory, namely a DRAM 935. As further seen, application processor 910 also couples to a capture device 945 such as one or more image capture devices that can record video and/or still images.

A universal integrated circuit card (UICC) 940 comprises a subscriber identity module, which in some embodiments includes a secure storage to store secure user information. System 900 may further include a security processor 950 (e.g., Trusted Platform Module (TPM)) that may couple to application processor 910. A plurality of sensors 925, including one or more multi-axis accelerometers may couple to application processor 910 to enable input of a variety of sensed information such as motion and other environmental information. In addition, one or more authentication devices may be used to receive, for example, user biometric input for use in authentication operations.

As further illustrated, a near field communication (NFC) contactless interface 960 is provided that communicates in an NFC near field via an NFC antenna 965. While separate antennae are shown, understand that in some implementations one antenna or a different set of antennae may be provided to enable various wireless functionalities.

A power management integrated circuit (PMIC) 915 couples to application processor 910 to perform platform level power management. To this end, PMIC 915 may issue power management requests to application processor 910 to enter certain low power states as desired. Furthermore, based on platform constraints, PMIC 915 may also control the power level of other components of system 900.

To enable communications to be transmitted and received such as in one or more internet of things (IoT) networks, various circuits may be coupled between baseband processor 905 and antenna 990. Specifically, a radio frequency (RF) transceiver 970 and a wireless local area network (WLAN) transceiver 975 may be present. In general, RF transceiver 970 may be used to receive and transmit wireless data and calls according to a given wireless communication protocol such as 5G wireless communication protocol such as in accordance with a code division multiple access (CDMA), global system for mobile communication (GSM), long term evolution (LTE) or other protocol. In addition, a GPS sensor 980 may be present, with location information being provided to security processor 950. Other wireless communications such as receipt or transmission of radio signals (e.g., AM/FM) and other signals may also be provided. In addition, via WLAN transceiver 975, local wireless communications, such as according to a Bluetooth™ or IEEE 802.11 standard can also be realized.

FIG. 22 shows a block diagram of a system in accordance with another embodiment of the present invention. Multiprocessor system 1000 is a point-to-point interconnect system such as a server system, and includes a first processor 1070 and a second processor 1080 coupled via a point-to-point interconnect 1050. Each of processors 1070 and 1080 may be multicore processors such as SoCs, including first and second processor cores (i.e., processor cores 1074a and 1074b and processor cores 1084a and 1084b), although potentially many more cores may be present in the processors. In addition, processors 1070 and 1080 each may include power controller unit 1075 and 1085. In addition, processors 1070 and 1080 each may include a secure engine to perform security operations such as attestations, IoT network onboarding or so forth.

First processor 1070 further includes a memory controller hub (MCH) 1072 and point-to-point (P-P) interfaces 1076 and 1078. Similarly, second processor 1080 includes a MCH 1082 and P-P interfaces 1086 and 1088. MCH's 1072 and 1082 couple the processors to respective memories, namely a memory 1032 and a memory 1034, which may be portions of main memory (e.g., a DRAM) locally attached to the respective processors. First processor 1070 and second processor 1080 may be coupled to a chipset 1090 via P-P interconnects 1062 and 1064, respectively. Chipset 1090 includes P-P interfaces 1094 and 1098.

Furthermore, chipset 1090 includes an interface 1092 to couple chipset 1090 with a high-performance graphics engine 1038, by a P-P interconnect 1039. In turn, chipset 1090 may be coupled to a first bus 1016 via an interface 1096. Various input/output (I/O) devices 1014 may be coupled to first bus 1016, along with a bus bridge 1018 which couples first bus 1016 to a second bus 1020. Various devices may be coupled to second bus 1020 including, for example, a keyboard/mouse 1022, communication devices 1026 and a data storage unit 1028 such as a non-volatile storage or other mass storage device. As seen, data storage unit 1028 may include code 1030, in one embodiment. As further seen, data storage unit 1028 also includes a trusted storage 1029 to store sensitive information to be protected. Further, an audio 1/O 1024 may be coupled to second bus 1020.

FIG. 23 depicts an IoT environment that may include wearable devices or other small form factor IoT devices. In one particular implementation, wearable module 1300 may be an Intel® Curie™ module that includes multiple components adapted within a single small module that can be implemented as all or part of a wearable device. As seen, module 1300 includes a core 1310 (of course in other embodiments more than one core may be present). Such a core may be a relatively low complexity in-order core, such as based on an Intel Architecture® Quark™ design. In some embodiments, core 1310 may implement a Trusted Execution Environment (TEE). Core 1310 couples to various components including a sensor hub 1320, which may be configured to interact with a plurality of sensors 1380, such as one or more biometric, motion, environmental or other sensors. A power delivery circuit 1330 is present, along with a non-volatile storage 1340. In an embodiment, this circuit may include a rechargeable battery and a recharging circuit, which may in one embodiment receive charging power wirelessly. One or more input/output (IO) interfaces 1350, such as one or more interfaces compatible with one or more of USB/SPI/I2C/GPIO protocols, may be present. In addition, a wireless transceiver 1390, which may be a Bluetooth™ low energy or other short-range wireless transceiver is present to enable wireless communications as described herein. In different implementations a wearable module can take many other forms. Wearable and/or IoT devices have, in comparison with a typical general-purpose CPU or a GPU, a small form factor, low power requirements, limited instruction sets, relatively slow computation throughput, or any of the above.

Embodiments may be used in many different types of systems. For example, in one embodiment a communication device can be arranged to perform the various methods and techniques described herein. Of course, the scope of the present invention is not limited to a communication device, and instead other embodiments can be directed to other types of apparatus for processing instructions, or one or more machine readable media including instructions that in response to being executed on a computing device, cause the device to carry out one or more of the methods and techniques described herein.

Program instructions may be used to cause a general-purpose or special-purpose processing system that is programmed with the instructions to perform the operations described herein. Alternatively, the operations may be performed by specific hardware components that contain hardwired logic for performing the operations, or by any combination of programmed computer components and custom hardware components. The methods described herein may be provided as (a) a computer program product that may include one or more machine readable media having stored thereon instructions that may be used to program a processing system or other electronic device to perform the methods or (b) at least one storage medium having instructions stored thereon for causing a system to perform the methods. The term “machine readable medium” or “storage medium” used herein shall include any medium that is capable of storing or encoding a sequence of instructions (transitory media, including signals, or non-transitory media) for execution by the machine and that cause the machine to perform any one of the methods described herein. The term “machine readable medium” or “storage medium” shall accordingly include, but not be limited to, memories such as solid-state memories, optical and magnetic disks, read-only memory (ROM), programmable ROM (PROM), erasable PROM (EPROM), electrically EPROM (EEPROM), a disk drive, a floppy disk, a compact disk ROM (CD-ROM), a digital versatile disk (DVD), flash memory, a magneto-optical disk, as well as more exotic mediums such as machine-accessible biological state preserving or signal preserving storage. A medium may include any mechanism for storing, transmitting, or receiving information in a form readable by a machine, and the medium may include a medium through which the program code may pass, such as antennas, optical fibers, communications interfaces, and the like. Program code may be transmitted in the form of packets, serial data, parallel data, and the like, and may be used in a compressed or encrypted format. Furthermore, it is common in the art to speak of software, in one form or another (e.g., program, procedure, process, application, module, logic, and so on) as taking an action or causing a result. Such expressions are merely a shorthand way of stating that the execution of the software by a processing system causes the processor to perform an action or produce a result.

A module as used herein refers to any hardware, software, firmware, or a combination thereof. Often module boundaries that are illustrated as separate commonly vary and potentially overlap. For example, a first and a second module may share hardware, software, firmware, or a combination thereof, while potentially retaining some independent hardware, software, or firmware. In one embodiment, use of the term logic includes hardware, such as transistors, registers, or other hardware, such as programmable logic devices. However, in another embodiment, logic also includes software or code integrated with hardware, such as firmware or micro-code.

### EXAMPLE SET 1

Example 1. A medical transducer for non-invasively and simultaneously heating the surface of an electrically conductive implant or a prosthesis located within or attached to a patient's body using magnetic induction; wherein the medical transducer is entirely external to (or outside of) the patient's body; and wherein the medical transducer is configured for applying a varying magnetic field (VMF) such as an alternating magnetic field (AMF) through only a portion of the patient's body around said implant or prosthesis, such as less than 20%, less than 50%, or less than 90% by volume of the entire patient's body.

Example 2. The medical transducer according to Example 1, wherein said implant or prosthesis is any implant within, or attached to, a human or animal body such as a knee implant, a hip implant, a shoulder implant, or an elbow implant, screws, plates, nails, and pins etc.

Example 3. The medical transducer according to Example 1, wherein said implant or prosthesis, such as a knee implant, includes one or more curved parts at least partially curving around a void space (optionally filled with bone or tissue) with two open ends, and wherein the medical transducer is so configured or positioned that at least a part of the flux flow of the VMF passes though said void space from one open end to the other.

Example 4. The medical transducer according to Example 3, which includes a housing with a semicylinder shape for harboring said portion of the patient's body, wherein the housing includes a first side compartment for accommodating a first coil, a second side compartment for accommodating a second coil, and a connecting compartment for connecting the two side compartments and for accommodating other components of the medical transducer.

Example 5. The medical transducer according to Example 4, wherein the first coil and the second coil are either symmetrical or asymmetrical relative a conceptual mirror plane.

Example 6. A method of designing the coil winding pattern to generate varying magnetic fields (VMF) such as alternating magnetic fields (AMF) from a medical transducer according to Example 1, comprising: (1) providing an electrically conductive implant or prosthesis (hereinafter “implant”) with a predetermined shape and predetermined material composition(s) throughout said implant; (2) determining a target current density/distribution or heat source distribution across the surface of said implant; (3) defining the shape of a surface of the medical transducer and simulating a continuous source current density distribution; (4) optimizing said source current density distribution of the medical transducer for generating a magnetic field distribution that can accomplish said target current density/distribution or heat source distribution on said implant; and (5) discretizing the optimized source current density distribution from step (4) into a winding pattern that can produce approximately the same magnetic field distribution.

Example 7. The method according to Example 6, wherein an objective function is defined in step (2) for producing a specified current density distribution J_{target}(x) on the surface of the implant:

$${f}_{R M S} ≡ {[ \frac{\int {\int }_{S} {( J ( x ) - {J}_{t a r g e t} ( x ) )}^{2} d S}{\int {\int }_{S} d S} ]}^{0.5} [ \frac{\int {\int }_{S} d S}{\int {\int }_{S} {J}_{t a r g e t ( x ) d S}} ] \qquad ( 1 ’ )$$

wherein J(x) is the magnitude of the current density/distribution at location x and S represents the surface of the implant, and the method aims to minimize the value of the objective function f_{RMS} so as to more closely match the target current density distribution.

Example 8. The method according to Example 7, wherein the implant components are made of different materials, and wherein different target current densities are applied on the components using said objective function.

Example 9. The method according to Example 7, wherein effects of spatial constraints are included in said objective function, and, if a constraint is breached, penalty terms are added to rapidly increase said objective function.

Example 10. The method according to Example 9, wherein said constraints comprise maximum and minimum allowable current densities or maximum and minimum heating rates.

Example 11. The method according to Example 6, wherein the interaction of the magnetic field with the implant before step (3) is modeled using the Maxwell-Ampere equation in the frequency domain:

$$∇ \times H = σ E + j ωD + {J}_{e} \qquad ( 2 ’ )$$

wherein H is the magnetic field, σ is the electrical conductivity, E is the electric field, ω is the angular frequency, D is the displacement field, and Jₑ is an external source current; and wherein Jₑ is the source current density distribution to be optimized.

Example 12. The method according to Example 11, wherein the optimization process is performed on a vector potential ψ instead of Jₑ, based on the following rationales:
(i) the source current density distribution is always divergence-free:

$$∇ \cdot {J}_{e} = 0 \qquad ( 3 ’ )$$

(ii) Jₑ is calculated from a vector potential ψ according to the following equation:

$$∇ \times Ψ = {J}_{e} \qquad ( 4 ’ )$$

(iii) since the divergence of the curl of a vector is always zero, the divergence of Jₑ is zero for any vector ψ:

$$∇ \cdot {J}_{e} = ∇ \cdot ( ∇ \times Ψ ) = 0. \qquad ( 5 ’ )$$

Example 13. The method according to Example 12, wherein the continuous source current density distribution in step (3) is confined within a specified volume or on a specified surface such as a cylindrical shell.

Example 14. The method according to Example 13, wherein the source current distribution is applied on a collection of surfaces (a shell), and the Jₑ term in the Maxwell-Ampere equation is replaced by a boundary condition on the shell:

$$n \times ( {H}_{1} - {H}_{2} ) = ( n \times {J}_{s} ) \times n \qquad ( 6 ’ )$$

wherein the magnetic field is discontinuous across the shell, and H₁ and H₂ in equation 6′ are the magnetic field on each side of the shell; the vector Jₛ is the applied surface current density on the shell; and n is the surface normal vector.

Example 15. The method according to Example 13, wherein the source current distribution is applied within a specified volume; wherein the surface current source is divergence-free; wherein the vector potential is replaced by the product of a scalar potential ψ and the surface normal vector:

$$∇ \times ( ψ n ) = {J}_{s} \qquad ( 7 ’ )$$

wherein Jₛ is constrained to be tangential to the edge at the outer edges of the shell to maintain the global conservation of current on the shell:

$$t \times {J}_{s} = 0 \qquad ( 8 ’ )$$

wherein t is the edge tangent unit vector; wherein, for a continuous surface, ∇×n=0, equation 7′ is rewritten as

$${J}_{s} = ∇ \times ( ψ n ) = ψ ∇ \times n + ( ∇ ψ ) \times n = ( ∇ ψ ) \times n \qquad ( 9 ’ )$$

and equation 8′ is then rewritten as

$$t \times [ ( ∇ ψ ) \times n ] = 0 \qquad ( 10 ’ )$$

wherein ψ is set to a constant value around the outer edges of the shell to full fill this condition.

Example 16. The method according to Example 7, wherein the finite element method is employed to solve the mathematical model of the physical system, and to find the optimal source current distribution on a shell by minimizing the objective function f_{RMS} in equation 1′.

Example 17. The method according to Example 16, wherein control variables are applied at each node point in the finite element mesh along the shell;
wherein said control variables represent the value of a scalar potential ψ at each node point, and
wherein the value of ψ between the node points is interpolated using standard finite element techniques.

Example 18. The method according to Example 17, wherein a gradient-based optimization solver to optimize said control variables.

Example 19. The method according to Example 18, wherein said gradient-based optimization solver is Sparse Nonlinear Optimizer (SNOPT).

Example 20. The method according to Example 18, wherein upper and lower limits are imposed on the value of w to reduce optimization space.

Example 21. The method according to Example 6, wherein equipotential lines are discretized into current loops in the execution of step (5): discretizing the optimized source current density distribution into a winding pattern.

Example 22. The method according to Example 15, wherein the source current density distribution is calculated from equation 11′:

$${J}_{s} = ( ∇ {ψ}_{f} ) \times n \qquad ( 11 ’ )$$

for any continuous surface;
wherein Jₛ is orthogonal to the surface normal vector (i.e., it is tangential to the surface);
wherein Jₛ is orthogonal to the gradient of ψ_{f}

$${J}_{s} \cdot ∇ {ψ}_{f} = 0 \qquad ( 12 ’ )$$

wherein the Jₛ vector is aligned with the direction of constant ψ_{f} at every point, and the current follows the contour lines of ψ_{f}.

Example 23. The method according to Example 22, wherein ψ_{f} is constrained to a constant value around the outer edges of a shell, and
wherein said equipotential contour lines form loops on the shell surfaces.

Example 24. The method according to Example 23, further comprising a step of optimizing the number of, and the distances between, said loops, to optimize the resistance of the windings and the total current traveling through the coil.

Example 25. The method according to Example 6, wherein said implant, such as a knee implant, includes one or more curved parts at least partially curving around a void space (optionally filled with bone or tissue) with two open ends, and wherein the medical transducer is so configured or positioned that a part of the flux flow of the VMF passes though said void space from one open end to the other.

Example 26. A method comprising: defining: (a) an implant's shape and material composition; (b) a sheet surface shape, and (c) an air domain to exist between the implant and the sheet surface (FIG. 10, block 1001) when alternating magnetic fields (AMF) are applied to the implant; determining a current density for the sheet surface (FIG. 10, block 1002); determining: (a) a dielectric value for at least one coil that couples to the sheet surface, (b) a dielectric value for the air domain, and (c) a dielectric value for the implant (FIG. 10, block 1004); determining a frequency for the AMF (FIG. 10, block 1003); in response to defining the implant's shape and material composition, defining at least one finite element model (FEM) for the implant, the sheet surface, and the air domain (FIG. 10, block 1003); determining at least one constraint, wherein the at least one constraint includes at least one of a current amplitude range for the at least one coil, a current direction for current to be transmitted by the at least one coil, and a spacing between adjacent portions of the at least one coil (FIG. 10, block 1007); determining an induced current distribution for the implant and an objective function (FIG. 10, block 1005); in response to determining an induced current distribution for the implant and an objective function, embedding the FEM in an optimization routine to minimize the objective function (FIG. 10, block 1006); determining a pattern for the at least one coil in response to: (a) determining the at least one constraint, (b) determining the frequency for the magnetic field, (c) determining the induced current distribution for the implant, and (d) embedding the FEM in the optimization routine.

As used herein, “defining: (a) an implant's shape” may include creation of a CAD model or the importation of a CAD model. Thus, Example 1 does not necessarily require creation of a CAD model (or any other model) but may allow for loading of a previously created model into a computing systems memory.

As such, an embodiment calculates a function based on the current density/distribution on the surface of a metal implant, rather than magnetic flux density.

An embodiment differs from pre-existing technologies in various ways. First, an embodiment is not a process for developing an optimization device. Instead, the embodiment uses an optimization method to obtain a coil pattern (on a current sheet) that can achieve a target (heat source/current density/distribution) on a desired object (e.g., an implant). Second, an embodiment is specifically designed to achieve uniform induction heating on an object of any shape and material composition. Third, an embodiment is not optimizing the shape of any object. Instead, the embodiment includes a generalized approach in such a way that the embodiment can achieve a coil pattern on any external shape (current sheet) that is desired. Fourth, in an embodiment the coil patterns obtained from optimization generate magnetic fields that can reproduce the value achieved on the object from optimization. Fifth, an embodiment uses a predefined objective function based on RMS, which differs from, for example, using an annealing method to optimize the objective function.

Example 27. The method of Example 26, wherein: the pattern for the at least one coil is configured to generate the AMF; the AMF is non-uniform and varies through the air domain.

Example 28. The method according to any of Examples 26 to 27, wherein the implant's shape is asymmetric in at least one of a sagittal plane, a coronal plane, or a transverse plane when the implant is implanted in a patient.

Example 29. The method according to any of Examples 26 to 28, wherein the implant's material composition includes at least two materials having differing chemical compositions from one another.

Example 30. The method according to any of Examples 26 to 29, wherein the current density for the sheet surface is a vector potential (FIG. 10, block 1002).

Example 31. The method according to any of Examples 26 to 30, wherein defining an implant's shape includes determining a three-dimensional computer aided design (CAD) model.

Example 32. The method according to any of Examples 26 to 31 comprising defining the at least one FEM for the implant, the sheet surface, and the air domain in response to defining at least one mesh for the implant, the sheet surface, and the air domain.

Example 33. The method according to any of Examples 26 to 32 comprising reducing the pattern for the at least one coil into at least one machine readable medium comprising a plurality of instructions that in response to being executed on a computing device, cause the computing device to render the pattern for the at least one coil.

For example, in some embodiments a digital pattern is the end product. An orthopedic prosthetic manufacturer may provide a new knee. An embodiment may analyze the knee and produce a coil pattern (and possibly transducer settings that work well with the coil pattern) and then deliver or communicate code or logic (i.e., an instruction set) to a coil manufacturer that can produce a physical coil. The instruction set is something that immediately leads to manufacture of a physical creation—a coil that improves biofilm eradication and improves the way in which the transducer/coil system operates. The improvements include a more efficient use of power for treating the biofilm and may also reduce the treatment time needed to treat a patient (which would further reduce power consumption). Thus, there is a more efficient use or resources. The “reverse process” from which the instruction set is derived is, among other things, novel and not well known to those of ordinary skill in the art. The software with the coil pattern improves the performance of the transducer/coil system.

Example 34. The method of Example 33 comprising physically coupling the at least one coil to the sheet surface.

Example 35. The method according to any of Examples 26 to 34, wherein: the sheet surface includes a long axis; a plane is orthogonal to the long axis; the at least one coil intersects the plane more than four times.

For example, the sheet surface of FIG. 15(B) includes a long axis (not shown) that runs vertically in the figure. A plane orthogonal to the axis may be situated to intersect the sheet about one half way up or down (vertically) the sheet. Such a plane may intersect the visible coil portions shown in FIG. 15(B) nine or more times.

Example 36. The method of Example 35, wherein: an additional axis is orthogonal to the long axis; the at least one coil intersects the additional axis more than three times.

For example, the coil may be stacked upon itself in layers.

Example 37. The method according to any of Examples 26 to 36, wherein the coil pattern is asymmetric.

Example 38. The method according to any of Examples 26 to 37, wherein the coil pattern includes a crescent shaped portion.

For example, the pattern of FIG. 15(B) includes a generally crescent or “C” shaped pattern despite the fact the portion of the at least one coil is not a perfect “C” and not all portions of the at least one coil are in the “C” shape.

Example 39. The method of Example 38, wherein the implant is a knee implant.

Example 40. The method according to any of Examples 26 to 39, wherein the sheet surface is a truncated cylinder.

For example, a cylindrical segment, sometimes also called a truncated cylinder, is the solid cut from a circular cylinder by two (or more) planes.

Example 41. The method according to any of Examples 26-40, wherein: the sheet surface includes a long axis; a plane is orthogonal to the long axis; the sheet surface is configured to be placed over a patient's limb; when in use, the plane intersects the sheet surface and the patient's limb.

Example 42. The method according to any of Examples 26-41 comprising: coupling the at least one coil to a current source; generating the AMF to prevent or treat biofilm on the implant.

Another version of Example 42. The method according to any of Examples 26-41 comprising: coupling the at least one coil to a current source; generating the AMF.

Example 43. The method of Example 42, wherein the current source is divergence free.

Example 44. At least one machine readable medium comprising a plurality of instructions that in response to being executed on a computing device, cause the computing device to carry out a method according to any one of Examples 26 to 43.

Example 45. An apparatus comprising means for performing any one of Examples 26 to 43.

Example 46. A system comprising: a sheet surface shape partially inclosing an air domain; at least one coil coupled to the sheet surface; wherein: the sheet surface includes a long axis; a plane is orthogonal to the long axis; the at least one coil intersects the plane more than four times.

Example 47. The system of Example 46, wherein: an additional axis is orthogonal to the long axis; the at least one coil intersects the additional axis more than three times.

Example 48. The system according to any of Examples 46 to 47, wherein the coil pattern is asymmetric.

Example 49. The system according to any of Examples 46 to 48, wherein the coil pattern includes a crescent shaped portion.

Example 50. The system according to any of Examples 46 to 49, wherein the sheet surface is a truncated cylinder.

Example 51. The system according to any of Examples 46-50, wherein: the sheet surface includes a long axis; a plane is orthogonal to the long axis; the sheet surface is configured to be placed over a patient's limb; when in use, the plane intersects the sheet surface and the patient's limb.

Example 52. The system according to any of Examples 46-51, wherein: the at least one coil intersects the plane more than four times at at least four locations; the spacing between adjacent locations of the at least four locations is irregular.

For example, in FIG. 15(B) there are locations where directly adjacent coil portions are closely located to one another and other locations where the spacing between directly adjacent coil portions is more spread out from one another.

Example 53. The system according to any of Examples 46-52, wherein the coil pattern is an open shape comprised of curvilinear segments.

An open shape is made up of line segments, but there is at least one line segment that isn't connected to anything at one of its endpoints. The shape is not a closed figure. If a shape is enclosed from all the sides end-to-end and form a figure with no openings is called a closed shape.

Example 54. The system of Example 53, wherein the coil pattern does not include more than two straight segments.

Example 55. A computer-implemented method for developing an optimized coil for the generation of an electromagnetic field to uniformly heat a conductive implant in man or any animal, wherein: (1) the initial input comprises the definition of a surface where the coil will be located, a 3D CAD model of the implanted item (including its material parameters), and a function that is evaluated to determine the optimized heating, (2) the current density required for optimized heating is defined as a vector potential, (3) the evaluation comprises: (i) computing an optimum distribution of current on the winding surface, (ii) comparing the target objective quantity of current on the implant with the computed valued, and (iii) generating iterations of the current density calculation until the objective function is satisfied.

The current density calculated on the surface is then reduced to physically realizable coils. The method then models the coils, and calculates the induced heating in the implant to verify optimum implant heating,

### EXAMPLE SET 2

Example 1. A method comprising:
determining a target electric current across a surface of an orthopedic implant;
in response to determining the target electric current across the surface of the orthopedic implant, determining an electric current pattern for a transducer coil winding;
optimizing the electric current pattern for the transducer coil winding to generate a magnetic field to induce actual electric current across the surface of the orthopedic implant, wherein the actual electric current is within a predetermined variance of the target electric current;
in response to optimizing the electric current pattern for the transducer coil winding, producing a coil pattern for the transducer coil winding;
forming the transducer coil winding configured in the coil pattern.

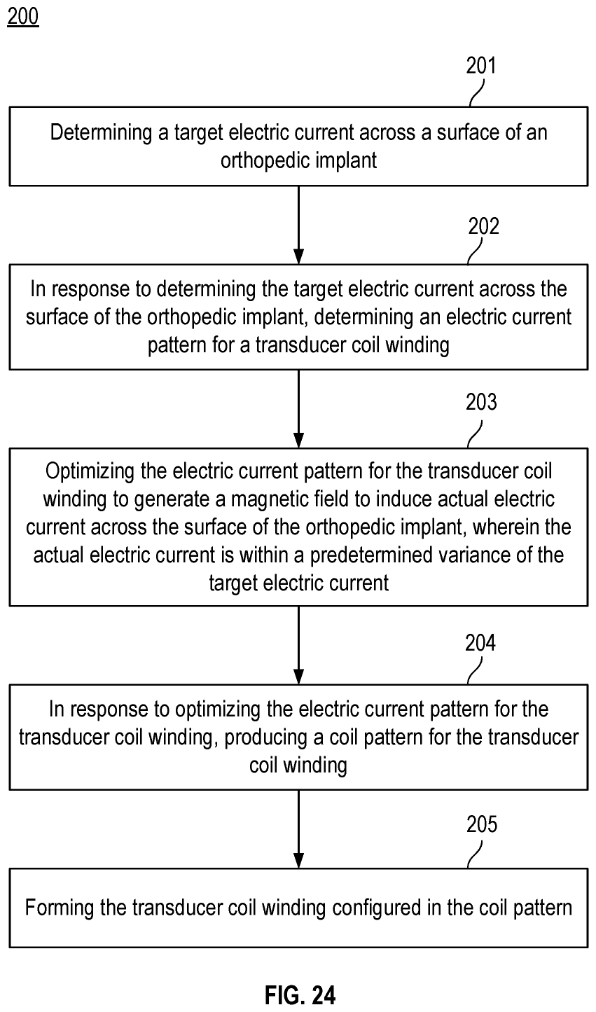

FIG. 24 includes process 200. Block 201 includes determining a target electric current across a surface of an orthopedic implant. Block 202 includes in response to determining the target electric current across the surface of the orthopedic implant, determining an electric current pattern for a transducer coil winding. Block 203 includes optimizing the electric current pattern for the transducer coil winding to generate a magnetic field to induce actual electric current across the surface of the orthopedic implant, wherein the actual electric current is within a predetermined variance of the target electric current. Block 204 includes in response to optimizing the electric current pattern for the transducer coil winding, producing a coil pattern for the transducer coil winding. Block 205 includes forming the transducer coil winding configured in the coil pattern.

Example 2. The method of Example 1, wherein:
the target electrical current includes at least one of current density, current distribution, or combinations thereof;
the actual electric current includes at least one of current density, current distribution, or combinations thereof.

Example 3. The method according to any of Examples 1-2 comprising:
coupling the transducer coil winding to a current source;
supplying current from the current source to the transducer coil winding to generate an alternating magnetic field (AMF) to treat a biofilm.

Example 4. The method according to any of Examples 1-3 comprising:
determining a three-dimensional computer aided design (CAD) model for the orthopedic implant;
in response to determining the three-dimensional CAD model for the orthopedic implant, determining the electric current pattern for the transducer coil winding.

Example 5. The according to any of Examples 1-4, wherein the optimizing the electric current pattern for the transducer coil winding to generate the magnetic field to induce actual electric current across the surface of the orthopedic implant includes optimizing the electric current pattern for the transducer coil winding to generate the magnetic field to induce uniform thermal heating across the surface of the orthopedic implant.

Example 6. The method according to any of Examples 1-4, wherein the optimizing the electric current pattern for the transducer coil winding to generate the magnetic field to induce actual electric current across the surface of the orthopedic implant includes optimizing the electric current pattern for the transducer coil winding to generate the magnetic field to induce uniform thermal heating across a majority of the surface of the orthopedic implant.

Example 7. The method according to any of Examples 1-6, wherein the orthopedic implant's shape is asymmetric in at least one of a sagittal plane, a coronal plane, or a transverse plane when the implant is implanted in a patient.

Example 8. The method according to any of Examples 1-7, wherein the orthopedic implant's material composition includes at least two materials having differing chemical compositions from one another.

Example 9. The method of Example 8, wherein:
determining the target electric current across the surface of an orthopedic implant includes determining differing target electric currents respectively for the differing chemical compositions;
the method further comprises in response to determining the differing target electric currents, determining differing electric current patterns for the transducer coil winding.

Example 10. The method according to any of Examples 1-9 comprising:
defining at least one finite element model (FEM) for the orthopedic implant;
in response to determining the FEM for the orthopedic implant, determining the electric current pattern for the transducer coil winding.

In alternative embodiments, methods other than FEM-based methods may be used such, without limitations, embodiments based on: (1) Boundary Element Method (BEM), (2) Finite Difference Method (FDM), (3) Finite Difference Time Domain (FDTD), (4) or combinations thereof.

Example 11. The method according to any of Examples 1-10, wherein the coil pattern for the transducer coil winding is asymmetric.

Example 11.1 The method according to any of Examples 1-11, wherein the coil pattern for the transducer coil winding is asymmetric in at least one of a sagittal plane, a coronal plane, or a transverse plane when the coil is in place on a patient.

Example 12. The method according to any of Examples 1-11.1, wherein the coil pattern for the transducer coil winding includes a crescent shaped portion.

Example 13. The method according to any of Examples 1-12, wherein the optimizing the electric current pattern for the transducer coil winding to generate a magnetic field to induce the actual electric current across the surface of the orthopedic implant includes optimizing the electric current pattern for the transducer coil winding to generate an alternating non-uniform magnetic field that varies through an air domain to induce the actual electric current across the surface of the orthopedic implant.

Example 14. The method according to any of Examples 1-13 comprising determining an objective function and, based on the objective function, optimizing the electric current pattern for the transducer coil winding to generate the magnetic field to induce the actual electric current across the surface of the orthopedic implant.

Example 15. The method of Example 14, wherein the objective function is a root means square (RMS) objective function.

Example 16. The method according to any of Examples 1-15, wherein the transducer coil winding is on a substrate and the method comprises optimizing the electric current pattern for the transducer coil winding to generate the magnetic field to induce actual electric current across the surface of the orthopedic implant based on a shape of the substrate.

Example 17. At least one machine readable medium comprising a plurality of instructions that in response to being executed on a computing device, cause the computing device to carry out a method according to any one of Examples 1 to 16.

Example 18. An apparatus comprising means for performing any one of Examples 1 to 16.

Many implants herein are referred to as orthopedic implants. However, embodiments are not so limited and may be applicable to implants more generally or any surface susceptible to biofilm.

The foregoing description of the embodiments of the invention has been presented for the purposes of illustration and description. It is not intended to be exhaustive or to limit the invention to the precise forms disclosed. This description and the claims following include terms, such as left, right, top, bottom, over, under, upper, lower, first, second, etc. that are used for descriptive purposes only and are not to be construed as limiting. For example, terms designating relative vertical position refer to a situation where a side of a substrate is the “top” surface of that substrate; the substrate may actually be in any orientation so that a “top” side of a substrate may be lower than the “bottom” side in a standard terrestrial frame of reference and still fall within the meaning of the term “top.” The term “on” as used herein (including in the claims) does not indicate that a first layer “on” a second layer is directly on and in immediate contact with the second layer unless such is specifically stated; there may be a third layer or other structure between the first layer and the second layer on the first layer. The embodiments of a device or article described herein can be manufactured, used, or shipped in a number of positions and orientations. Persons skilled in the relevant art can appreciate that many modifications and variations are possible in light of the above teaching. Persons skilled in the art will recognize various equivalent combinations and substitutions for various components shown in the Figures. It is therefore intended that the scope of the invention be limited not by this detailed description, but rather by the claims appended hereto.

1. A method comprising:
determining a target electric current across a surface of an orthopedic implant;
in response to determining the target electric current across the surface of the orthopedic implant, determining an electric current pattern for a transducer coil winding;
optimizing the electric current pattern for the transducer coil winding to generate a magnetic field to induce actual electric current across the surface of the orthopedic implant, wherein the actual electric current is within a predetermined variance of the target electric current;
in response to optimizing the electric current pattern for the transducer coil winding, producing a coil pattern for the transducer coil winding;
forming the transducer coil winding configured in the coil pattern.

2. The method of claim 1, wherein:
the target electric current includes at least one of current density, current distribution, or combinations thereof;
the actual electric current includes at least one of current density, current distribution, or combinations thereof.

3. (canceled)

4. The method of claim 2 comprising:
determining a three-dimensional computer aided design (CAD) model for the orthopedic implant;
in response to determining the three-dimensional CAD model for the orthopedic implant, determining the electric current pattern for the transducer coil winding.

5-18. (canceled)

19. At least one non-transitory machine-readable medium having stored thereon data, which if used by at least one machine having at least one processor and at least one input/output (I/O) channel, causes the at least one machine to perform a method comprising:
determining a target electric current across a surface of an orthopedic implant,
in response to determining the target electric current across the surface of the orthopedic implant, determining an electric current pattern for a transducer coil winding;
optimizing the electric current pattern for the transducer coil winding to generate a magnetic field to induce actual electric current across the surface of the orthopedic implant, wherein the actual electric current is within a predetermined variance of the target electric current;
in response to optimizing the electric current pattern for the transducer coil winding, producing a coil pattern for the transducer coil winding.

20. The at least one medium of claim 19, wherein:
the target electric current includes at least one of current density, current distribution, or combinations thereof;
the actual electric current includes at least one of current density, current distribution, or combinations thereof.

21. The at least one medium of claim 20, the method comprising producing an instruction set corresponding to the coil pattern for the transducer coil winding, which if used by at least one additional machine having at least one additional processor, causes the at least one additional machine to form a physical transducer coil winding having the coil pattern.

22. The at least one medium of claim 21, the method comprising storing the instruction set in the at least one non-transitory machine-readable medium and communicating the instruction set to a user via the at least one I/O channel.

23. The at least one medium of claim 20, wherein the optimizing the electric current pattern for the transducer coil winding to generate the magnetic field to induce actual electric current across the surface of the orthopedic implant includes optimizing the electric current pattern for the transducer coil winding to generate the magnetic field to induce uniform thermal heating across the surface of the orthopedic implant.

24. The at least one medium of claim 20, wherein the optimizing the electric current pattern for the transducer coil winding to generate the magnetic field to induce actual electric current across the surface of the orthopedic implant includes optimizing the electric current pattern for the transducer coil winding to generate the magnetic field to induce uniform thermal heating across a majority of the surface of the orthopedic implant.

25. The at least one medium of claim 24, wherein the orthopedic implant has a shape that is asymmetric in at least one of a sagittal plane, a coronal plane, or a transverse plane when the implant is implanted in a patient.

26. The at least one medium of claim 25, wherein the orthopedic implant's material composition includes at least two materials having differing chemical compositions from one another.

27. The at least one medium of claim 26, wherein:
determining the target electric current across the surface of an orthopedic implant includes determining differing target electric currents respectively for the differing chemical compositions;
the method further comprises in response to determining the differing target electric currents, determining differing electric current patterns for the transducer coil winding.

28. The at least one medium of claim 19, the method comprising:
defining at least one finite element model (FEM) for the orthopedic implant;
in response to determining the FEM for the orthopedic implant, determining the electric current pattern for the transducer coil winding.

29. The at least one medium of claim 19, wherein the coil pattern for the transducer coil winding is asymmetric.

30. The at least one medium of claim 19, wherein the coil pattern for the transducer coil winding includes a crescent shaped portion.

31. The at least one medium of claim 19, wherein the optimizing the electric current pattern for the transducer coil winding to generate a magnetic field to induce the actual electric current across the surface of the orthopedic implant includes optimizing the electric current pattern for the transducer coil winding to generate an alternating non-uniform magnetic field that varies through an air domain to induce the actual electric current across the surface of the orthopedic implant.

32. The at least one medium of claim 19, the method comprising determining an objective function and, based on the objective function, optimizing the electric current pattern for the transducer coil winding to generate the magnetic field to induce the actual electric current across the surface of the orthopedic implant.

33. The at least one medium of claim 32, wherein the objective function is a root means square (RMS) objective function.

34. The at least one medium of claim 19, wherein the transducer coil winding is on a substrate and the method comprises optimizing the electric current pattern for the transducer coil winding to generate the magnetic field to induce actual electric current across the surface of the orthopedic implant based on a shape of the substrate.

35. The at least one medium of claim 19, the method comprising:
determining a three-dimensional computer aided design (CAD) model for the orthopedic implant;
in response to determining the three-dimensional CAD model for the orthopedic implant, determining the electric current pattern for the transducer coil winding.
